(19) United States
(12) Patent Application Publication
(10) Pub. No.: US 2024/0230609 A1
(43) Pub. Date: Jul. 11, 2024

(54) POTASSIUM MALEATE BUFFER FOR FREE CHLORINE ANALYSIS

(71) Applicant: HACH COMPANY, Loveland, CO (US)

(72) Inventors: Douglas ROYER, Gilbert, IA (US); Melinda Buyck, Nevada, IA (US); Sarah Anciaux, Fort Collins, CO (US); Devin Maurer, Ankeny, IA (US)

(73) Assignee: HACH COMPANY, Loveland, CO (US)

(21) Appl. No.: 18/151,161

(22) Filed: Jan. 6, 2023

(51) Int. Cl. G01N 31/22; C07C 59/245; C07C 211/51; G01N 21/78

(52) U.S. Cl. CPC G01N 31/22; C07C 59/245; C07C 211/51; G01N 21/78

(57) ABSTRACT

A kit is described that can be used for measuring free chlorine in water. The kit includes a buffer with potassium maleate and an indicator. A method for measuring a concentration of free chlorine in a water system includes adding an indicator and a buffer with potassium maleate to a water sample collected from the water system, measuring a light absorbance of the water sample after the indicator and the buffer have been added to the water sample, and determining a concentration of the free chlorine in the water system based on the measured light absorbance.

### TECHNICAL FIELD

The present disclosure relates generally to a buffer that is used in measuring free chlorine in a water sample.

### BACKGROUND

Chlorine is commonly used as a disinfectant in water systems and wastewater treatment processes. Measuring and monitoring free chlorine is important for ensuring safe drinking water and meeting environmental regulatory requirements for wastewater and industrial streams.

Colorimetric assays can be used to measure free chlorine in water where an indicator and a buffer are added to a water sample. The indicator reacts with the free chlorine in the sample to produce a color having an absorbance proportional to the concentration of free chlorine. The absorbance at a particular wavelength can be measured to determine the free chlorine concentration. The buffer adjusts the pH of the sample to a range that is optimum for measurement of free chlorine.

A common buffer for free chlorine measurement is lithium maleate (C₄H₂Li₂O₄). However, lithium in the buffer can interfere with electrodes measuring the pH of the system. In addition, lithium can form insoluble particles with fluoride and phosphate, which are elements commonly found in water and wastewater streams. The turbidity from the insoluble particles can interfere with accurate colorimetric measurements of free chlorine. Lithium is also a psychoactive drug which is undesirable in water streams. Moreover, lithium hydroxide, for forming lithium maleate, is most commonly available in a powdered form, presenting an inhalation hazard.

### SUMMARY

In view of the above, an object of the present disclosure is to provide a buffer that reduces or eliminates the common drawbacks of lithium maleate as a buffer for measurement of free chlorine in water.

In one aspect, this disclosure provides a kit comprising a buffer including potassium maleate and an indicator for measuring free chlorine in water.

In another aspect, this disclosure provides a method of measuring a concentration of free chlorine in a water system, the method comprising adding an indicator and a buffer including potassium maleate to a water sample collected from the water system, measuring a light absorbance of the water sample after the indicator and the buffer have been added to the water sample, and determining a concentration of the free chlorine in the water system based on the measured light absorbance.

### DETAILED DESCRIPTION OF EMBODIMENTS

In the following description, numerous details are set forth to provide an understanding of the present disclosure. However, it may be understood by those skilled in the art that the compositions and methods of the present disclosure may be practiced without these details and that numerous variations or modifications from the described embodiments may be possible.

Broadly speaking, an embodiment of the present disclosure provides potassium maleate (C₄H₂K₂O₄ and/or C₄H₃KO₄) as a buffer for the measurement of free chlorine in a water sample, in particular the measurement of free chlorine by a colorimetric assay.

In connection with this disclosure, it has been discovered that potassium maleate is an effective buffer in free chlorine assays and does not suffer the same disadvantages as lithium maleate. Potassium maleate has good solubility in water and is effective for adjusting a pH of a water sample to an optimal range for measuring free chlorine. In addition, potassium maleate does not interfere with electrodes measuring the pH of the water sample. Potassium maleate also does not form insoluble particles with compounds commonly found in water streams, such as fluorides and sulfates, and thus causes less interference with colorimetric measurements to determine free chlorine. Potassium maleate is not a psychoactive drug. Moreover, potassium hydroxide, for forming potassium maleate, is available in a pelletized form which diminishes any inhalation hazards.

Some embodiments of the present disclosure may include a kit. The kit may comprise a buffer including potassium maleate in a first container and an indicator in a second container. Alternatively, the potassium maleate buffer and indicator may be in the same container. The containers may be sealed containers, such as powder pillows, sealed plastic bags, vials, ampules, pouches, test tubes, etc., containing the potassium maleate buffer and the indicator. The first and second containers can be packaged together in a third larger container (e.g., sealed pouch, box, etc.) and provided as a reagent kit for measuring free chlorine concentration in water.

The buffer may be an aqueous solution of potassium maleate. A concentration of the potassium maleate in the solution may be in a range from 1 to 4 mol/liter (M), from 2 to 3 M, or from 2.4 to 2.8 M. In some embodiments, the potassium maleate may remain soluble, even at a high concentration within one or more of the above ranges, at temperatures ranging from 0 to 350 Celsius, from 1 to 250 Celsius, from 3 to 150 Celsius.

The potassium maleate buffer may have a pH in a range of approximately 6.5 to 8, from 7 to 8, or from 7.3 to 7.6.

The indicator may include N,N-diethyl-p-phenylenediamine (DPD). A concentration of the DPD may be in a range from 0.02 to 0.5 M, from 0.05 to 0.25 M, or from 0.1 to 0.2 M.

The DPD may be provided in a powdered form in one container. An indicator solution may be provided in a separate container. The DPD and the indicator solution may be added together before use. Mixing of DPD and the indicator solution may be facilitated by swirling or rocking the container, either manually or automatically, using a stir bar, or any other suitable means. The indicator solution may include deionized water. The deionized water may include an acid to increase stability. The acid may be a mineral acid or an organic acid with a concentration ranging from 0.05 to 1.5 M, from 0.2 to 1 M, or from 0.25 to 0.5 M. Once DPD is added to the indicator solution, the resulting concentration of the DPD in the indicator solution may be in a range from 0.02 to 0.5 M, from 0.05 to 0.25 M, or from 0.1 to 0.2 M.

The present disclosure also relates to a method of measuring free chlorine by using the indicator and potassium maleate as a buffer. The method includes adding the indicator and the buffer to water to measure the free chlorine concentration. For example, the indicator and buffer may be added to a water sample that has been collected from a water system.

When the indicator is added to water for free chlorine measurement, at or near a neutral pH, free chlorine in the sample may immediately react with (e.g., oxidize) the indicator to form a color compound (effectively a dye). The amount of dye formed is generally proportional to an amount of free chlorine present in the water. For example, free chlorine in the water oxidizes DPD to form a Wurster dye with a pink/magenta color having an intensity proportional to the free chlorine content.

The indicator may be added to the water before or after the buffer is added to the water. Alternatively, the indicator and buffer may be added to the water at the same time. The indicator and the buffer may be added to the water separately or may be combined and added to the water together. Once the indicator and/or buffer have been added to the water, mixing of the water, indicator, and/or buffer may be facilitated by swirling or rocking the container, either manually or automatically, using a stir bar, or any other suitable means.

In some embodiments, the water sample may be at or near a neutral pH before the addition of the indicator and the buffer. For example, the water sample may have an initial pH ranging from approximately 4 to 10, from 7 to 8, or from 7.3 to 7.6. An initial alkalinity of the water sample may range from 0 to 1,000 ppm as CaCO₃, from 0 to 500 ppm as CaCO₃, or from 0 to 200 ppm as CaCO₃. Adding the indicator to the water sample often lowers the pH of the sample outside of an acceptable range for an accurate measurement of free chlorine. For example, an equilibrium of chlorine species in the water sample may be pH dependent. In addition, too high or too low of a pH may result in interferences in the measurement. An optimal pH range for measurement by the indicator may be approximately 5.5 to 7, from 6 to 6.8, or from 6.3 to 6.6. The potassium maleate buffer can stabilize fluctuations in the pH.

When the indicator and the potassium maleate buffer are added to the water sample, the pH of the resulting mixture may range from approximately 5.5 to 7, from 6 to 6.8, or from 6.3 to 6.6.

In some embodiments of the present disclosure, a sample size of approximately 12 milliliters (ml) of water to be measured may be utilized with approximately 10 microliters (μl) of the indicator and approximately 2 μl of the potassium maleate buffer for measurement of free chlorine in the water sample. In some embodiments, a sample size of approximately 2 ml of water to be measured may be utilized with 0.025 ml of indicator and 0.025 ml of the potassium maleate buffer. In some embodiments, the size of the water sample to be measured may range from 60 to 100 ml and may be utilized with 1 ml of indicator and 1 ml of the potassium maleate buffer.

For example, the potassium maleate buffer to be added to approximately 12 ml of a water sample may range from 0.5 to 10 μl, from 1 to 5 μl, or from 1.5 to 4 μl. The volume of the indicator to be mixed with approximately 12 ml of a water sample may range from 1 to 50 μl, from 4 to 30 μl, or from 8 to 20 μl. The ranges of the potassium maleate and the indicator may scale proportionally with the water sample size.

The method further includes measuring an absorbance of the water sample after the potassium maleate buffer and the indicator have been added to the water sample. The light absorbance of the water sample can be measured at a wavelength in a range of from 480 to 580 nm, from 500 to 560 nm, or at 530 nm, for example. The absorbance can be measured using, for example, a spectrophotometer, colorimeter, or any other known instrument suitable for measuring absorbance. The concentration of free chlorine in the water sample collected from a water system can be determined from the measured absorbance.

For example, the concentration of free chlorine can be determined by comparing the measured absorbance with a calibration curve obtained in advance by known methods. For example, the calibration curve can be obtained by measuring the absorbance of multiple standard solutions of equal volume having different known concentrations of free chlorine. Based on the measured absorbance values at different free chlorine concentrations, a curve of absorbance versus free chlorine concentration can be plotted to generate the calibration curve. The calibration curve can then be used to determine the free chlorine concentration of water samples containing an unknown amount of free chlorine by comparing the measured absorbance of the water sample with the calibration curve.

Alternatively, the spectrophotometer, colorimeter, or other instrument for measuring the absorbance may be programmed so as to automatically determine the free chlorine concentration based on the measured absorbance value using, for example, a calibration curve that has been saved in a memory of the instrument.

The present disclosure also relates to a method of making a potassium maleate buffer. The method may include forming maleic acid by reacting maleic anhydride with water. The maleic acid may then be reacted with potassium hydroxide to form dipotassium maleate and monopotassium maleate, together potassium maleate.

The foregoing is further illustrated by reference to the following examples, which are presented for purposes of illustration and are not intended to limit the scope of the present disclosure.

### EXAMPLES

### Example 1—Measurement of Free Chlorine in Water Samples

0.025 ml of a 0.1 M DPD indicator solution stabilized with 0.5 M organic acid, and 0.025 ml of a 2.7 M potassium maleate buffer were added to each of five samples including 2 ml of water containing free chlorine to be measured. The samples were assayed by a CL17 Analyzer manufactured by Hach Company to measure the free chlorine concentration based on the light absorbance at 530 nm. The average result for each sample and a standard deviation of the results are shown in Table 1. A standard assay was also performed to measure the free chlorine concentration in each of the samples.

TABLE 1

| | Standard Assay | CL17 Analyzer | |
|---|---|---|---|
| | Free Chlorine | Average | |
| | Concentration | Measured | Standard |
| Sample | (mg/L) | Result (mg/L) | Deviation |
| 1 | 4.37 | 4.40 | 0.063 |
| 2 | 2.87 | 2.90 | 0.005 |
| 3 | 1.78 | 1.81 | 0.002 |
| 4 | 1.09 | 1.12 | 0.003 |
| 5 | 0.59 | 0.61 | 0.003 |

The results shown in Table 1 demonstrate that the potassium maleate can be used as a buffer in a method for accurately measuring free chlorine concentration using an indicator.

Although some embodiments of the invention have been described in detail above, those skilled in the art will readily appreciate that many modifications are possible in the example embodiments without materially departing from the disclosed embodiments. Accordingly, all such modifications are intended to be included within the scope of this disclosure as defined in the following claims.

What is claimed is:

1. A kit comprising:
a buffer including potassium maleate; and
an indicator for measuring free chlorine in water.

2. The kit of claim 1, wherein a concentration of the potassium maleate in the buffer is in a range of 1 to 4 mol/liter (M).

3. The kit of claim 1, wherein the buffer includes an aqueous solution of the potassium maleate.

4. The kit of claim 3, wherein the potassium maleate is soluble in the aqueous solution at a temperature in a range of from 0° C. to 35° C.

5. The kit of claim 1, wherein the buffer has a pH in a range of 7 to 8.

6. The kit of claim 1, wherein the indicator is N,N-diethyl-p-phenylenediamine (DPD).

7. A method of measuring a concentration of free chlorine in a water system, the method comprising:
adding an indicator and a buffer including potassium maleate to a water sample collected from the water system;
measuring a light absorbance of the water sample after the indicator and the buffer have been added to the water sample; and
determining a concentration of the free chlorine in the water system based on the measured light absorbance.

8. The method of claim 7, wherein the water sample has a pH in a range of 4 to 10 before the indicator and the buffer are added.

9. The method of claim 7, wherein the water sample has a pH in a range of 5.5 to 7 after the indicator and the buffer are added.

10. The method of claim 7, wherein the buffer is produced by reacting potassium hydroxide with maleic acid.

11. The method according to claim 10, wherein the maleic acid is produced by reacting maleic anhydride is reacted with water.

12. The method according to claim 7, wherein the buffer includes dipotassium maleate and monopotassium maleate.

13. The method according to claim 7, wherein the indicator is N,N-diethyl-p-phenylenediamine (DPD).
